(19) United States
(12) Patent Application Publication
(10) Pub. No.: US 2026/0019679 A1
(43) Pub. Date: Jan. 15, 2026

(54) METHOD FOR USER-FRIENDLY PLAYBACK OF A VIDEO AND ASSOCIATED ENDOSCOPIC IMAGE RECORDING SYSTEM

(71) Applicant: Schölly Fiberoptic GmbH, Denzlingen (DE)

(72) Inventors: Daniel HÄRTER, Emmendingen (DE); Stefan SCHRÖER, Freiburg (DE); Alexander KÖHLER, Freiburg (DE); Sébastien WEITBRUCH, Niedereschach (DE)

(73) Assignee: Schölly Fiberoptic GmbH, Denzlingen (DE)

(21) Appl. No.: 19/261,311

(22) Filed: Jul. 7, 2025

(30) Foreign Application Priority Data
Jul. 10, 2024 (DE) 102024119577.0

(51) Int. Cl. H04N 21/472; A61B 1/00; A61B 1/04; A61B 1/06

(52) U.S. Cl. CPC H04N 21/47217; A61B 1/00045; A61B 1/043; A61B 1/06

(57) ABSTRACT

In summary, a simplified approach for user-friendly playback of a video (5) is provided, which was previously recorded using an endoscopic image recording system (1). To offer the user a high level of security in the use of the image recording system (1) and good usage properties at the same time, it is provided that in a replay mode (37), to which the user can change, for example, by a replay user input (22), on the one hand, a currently recorded live video image (6) is visualized using the image recording system (1), and at the same time the user can observe a playback of the previously recorded video (5) on a display device (8) of the system (1).

### CROSS-REFERENCE TO RELATED APPLICATIONS

This application claims priority to German Patent Application No. 10 2024 119 577.0, filed Jul. 10, 2024, which is incorporated herein by reference as if fully set forth.

### TECHNICAL FIELD

The invention relates to a method for playing back a video in a user-friendly manner, wherein the video was previously recorded using an endoscopic image recording system and wherein this image recording system can be designed as described herein. The video can visualize, for example, a flooding procedure carried out using a fluorophore in tissue. In addition, the invention is particularly advantageously usable if the video is recorded in an advanced imaging mode (“advanced imaging mode”) deviating from a white light imaging mode, which is used to display advanced image information (thus, for example, a fluorescence imaging mode or an infrared (IR) imaging mode).

The invention additionally relates to an endoscopic image recording system, which comprises an endoscope, at least one image sensor, and a memory, which can be designed as an external or internal memory, in particular in the form of a buffer memory. At least one image sensor of the system can preferably be configured in this case to record fluorescent light and/or advanced image information. The memory is used here to store videos, which are/were recorded using the at least one image sensor.

### BACKGROUND

One exemplary application in which the invention is relevant is formed by a typical operating situation, in which the body of a patient is already open and a fluorophore is introduced into a previously surgically treated organ or other tissue by means of a syringe by the operator, wherein the tissue/organ is irradiated using excitation light and with the aid of an endoscope, the fluorescence wavelengths generated by the fluorophore in reaction to the excitation are recorded using the endoscope in the scope of a fluorescent light imaging as fluorescence images. This is referred to here as a flooding procedure, because the fluorophore slowly spreads into all branches of the organ/the tissue via the blood vessels. This dynamic procedure typically lasts approximately one minute, wherein the operator initially can observe the spreading of the fluorophore in the larger vessels live on the display screen on the basis of the fluorescence signal recorded by means of the endoscope.

This type of “advanced imaging” is used by the operator/the surgeon for quality control of the previously performed surgical intervention: if it is shown, for example, upon recording the fluorescence images that a specific part of the organ/the tissue is not sufficiently perfused, the operator can remedy this circumstance by means of a surgical measure. It is thus in particular possible to avoid specific parts of the tissue only being supplied with blood inadequately and as a result possibly dying or becoming necrotic, which would make a further later surgical intervention necessary.

### SUMMARY

Proceeding therefrom, the invention is based on the object of enabling improved handling and operation of an endoscopic image recording system during surgical interventions for the operator.

To achieve this object according to the invention, in a method for playing back a video with the aid of an endoscopic image recording system, one or more of the features disclosed herein are provided. Therefore, to achieve the object according to the invention, in a method of the type mentioned at the outset, it is provided in particular that after the recording of the video, a change is made to a replay mode in which the video is displayed. This change can preferably take place automatically or in reaction to a replay user input, which a user actuates (for example, manually or via speech input or by means of a foot switch).

Furthermore, it is provided for the achievement that the video was/is stored in a memory of the image recording system and is loaded from this memory for playback in the replay mode. And finally, it is provided that in the replay mode a live video image just recorded using the image recording system is displayed by the image recording system simultaneously with the played back and previously recorded video.

This method can particularly develop its advantages if the live video image is a white light image and/or is currently captured (thus in the current situation) in a white light imaging mode (WLI mode) using the image recording system.

In the application mentioned at the outset, an operator, following the concept of the invention, can initially record a short video clip (of, for example, approximately one minute duration) in a fluorescence imaging mode using an endoscope of the image recording system. Subsequently, he can view this video in detail in the replay mode and in this case also view specific phases of the flooding procedure multiple times and/or in an enlarged representation on a display device of the image recording system and analyze them accurately. A very accurate and reliable supervision of the previous operation result, more precisely the perfusion of the treated tissue, is thus possible. At the same time, the operator always retains an overview about the present operation scene with the aid of the live video image during this, so that, for example, they can see bleeds arising and stop them.

By means of the invention, the operator can therefore obtain access quickly and in an uncomplicated manner to the previously recorded video, so that even during the operation, they can inspect time intervals of a flooding procedure or of other procedures which they have recorded in an “advanced imaging mode” in the video in detail, in particular repeatedly.

The operator can thus check piece by piece, for example, whether all areas of previously surgically treated tissue are sufficiently perfused. It is to be considered in this case that especially tissue parts which are permeated by very fine blood vessels are only flushed through with fluorophore at the end of the flooding procedure, so that these areas first display a relevant fluorescence signal at the end of the video, while other tissue parts, which are permeated with larger blood vessels, possibly already display a fading or already entirely disappearing fluorescence signal at this time. The operator therefore has to have access to the entire video in high time resolution, because they have to inspect different image areas very accurately depending on the different image recording time, in order to ensure that all parts of the tissue are sufficiently perfused, thus display a sufficiently high fluorescence signal at least once at any time within the video. On the basis of the video, which is recorded using parameters optimized for playback and is played back in the replay mode using optimized settings, the operator can therefore cognitively comprehend the flooding procedure in the tissue step-by-step and thus check the result of the prior surgical intervention in order to be able to preclude said risks.

Among other things, it is therefore characteristic for the operating philosophy according to the invention that the functional scope accessible for the surgeon, which they can change via operating elements, is intentionally restricted to those functions which are reasonable and necessary in the respective situation. This is because a complex manual navigation into submenus is avoided by the rapid change into the replay mode having preset playback parameters, which substantially simplifies the use. In particular, the surgeon can change at arbitrary times to the replay mode here, without losing the supervision over the operation region in this case, because they can still see this in the live video image. Therefore, the usage properties of the system are decisively improved.

An automatic change into the replay mode can take place, for example, as soon as the user ends the recording or the recording of the video ends automatically after passage of a defined time span or only later by means of a separate replay user input.

It is therefore in particular characteristic for the invention that the live video image is recorded live and played back live in a second imaging mode (or also: image recording mode), which differs from a first imaging mode, in which the video played back in the replay mode was previously recorded. The two imaging modes or image recording modes can differ in particular here with respect to the illumination used during the respective image recording and/or in the imaging path and/or in the image sensor which is used in the respective imaging mode.

The object can also be achieved according to the invention by further advantageous embodiments as described below and in the claims.

For example, the previously recorded video can have been captured using a first image sensor of the image recording system, while the live video image was or is captured using a second image sensor of the image recording system. In this case, the first image sensor can preferably be configured for advanced imaging, for example, fluorescent light imaging. The second image sensor, in contrast, can preferably be configured for white light imaging.

Alternatively to the use of at least two image sensors, however, the method can also provide that both the video and the later live video image are/were recorded using the same image sensor of the image recording system. In this case, the video is/was thus recorded using an image sensor of the image recording system, using which the live video image is also recorded during the replay mode.

In the first variant of the use of two spatially separated image sensors (or at least two spatially separated areas of a common image sensor surface), it is possible to use both imaging modes simultaneously, so that different types of images can be recorded simultaneously.

The previously recorded video and the live video image can be displayed in parallel playback either on separate display devices or on a common display device of a user. In the latter case, the video and the live video image can be displayed, for example, in a split-screen view or in a side-by-side view or in a picture-in-picture view. Parallel playback can thus be understood here in particular to mean that both video streams (thus the video retrieved from the memory and the live video image) can be played back simultaneously and therefore can be observed simultaneously by the user (either on a common display device or on two separate display devices).

Depending on the embodiment of the method, the previously recorded video can in particular be based on a sensorial capture of fluorescent light, which is emitted by a (in particular the abovementioned) fluorophore in reaction to an optical excitation using excitation light, which can take place in particular during a flooding procedure as described above.

However, it is also possible that the recorded video visualizes at least one item of advanced image information, which was calculated from sensorially captured image data. For example, it is possible to read out signals from individual color channels of the respective image sensor and offset them with one another so that a specific physical variable can be visualized, for example, the presence of a specific chromophore in the tissue. In this way, for example, a local blood oxygen saturation can be visualized, which is not directly accessible from the image data without such a calculation.

One particularly preferred embodiment of the method provides that the replay mode is one view mode of a series of different view modes, each of which a user can set at the image recording system by means of a view mode user input. For example, it can be provided that by repeatedly executing such a view mode user input, it is possible to change between the different view modes, which can preferably take place in a predetermined sequence. Particularly simple operation is possible, for example, if the individual view modes of the series can be selected in an endless loop by repeatedly executing the view mode user input.

Furthermore, it is preferred if it is possible to change between at least three different view modes of the series by means of an operating element and if these view modes comprise at least one white light view mode, at least one advanced view mode (which is based on an advanced imaging mode), and the mentioned replay mode. To improve the usage properties for the user, it can be provided here that the operating element is designed as manual. Furthermore, it is advantageous if the operating element is arranged at an endoscope of the image recording system.

Each of the mentioned view modes of the series can in this case comprise an imaging mode, which is presently used to generate the live video image, and a display mode in which the live video image is visualized on a display device. Moreover, each individual one of the view modes can define parameters of the respective imaging mode and the respective display mode.

It is furthermore advantageous if the white light view mode defines parameters of a white light imaging mode and the advanced view mode in turn defines parameters of an advanced imaging mode (thus in particular a fluorescence imaging mode or an imaging mode which enables a visualization of advanced image information such as a spatial distribution of oxygen saturation). Furthermore, the replay mode, as already mentioned, can define parameters of a white light imaging mode for the live video image and a display mode for parallel playback of the previously stored videos and the live video image at the same time.

It can furthermore be provided in the method that a user in the replay mode can navigate within the video by means of a navigation user input, in order to thus have different time intervals of the video displayed. For example, it can be provided that it is possible to jump forward or back in the video by a defined time span by means of the navigation user input. In this case, the video can preferably be played back during this in the replay mode in a video endless loop.

Instead of designing an operating element so that three different user inputs can be actuated using this operating element (for example, by presses of different lengths in time and/or by movement of the manual operating element in different directions (for example, pressing and sliding to the left or right)), it is also possible to form at least two operating elements in the handle area in order to be able to actuate the total of three desired different user inputs.

Further possible operating elements which can be used according to the invention for actuating user inputs are foot switches or other input devices which can be operated, for example, by assistance personnel in the operating room. Moreover, speech inputs are also conceivable and can be designed if the image recording system has a microphone. It is to be noted that all of the abovementioned possible user inputs are used to control the video, which is loaded from the internal memory of the image recording system and played back in the replay mode.

Of course, user inputs, using which the reproduction, the pausing, the fast forward, or the fast backward or other playback functions are changed, can therefore be input in a variety of ways in a known manner by a user. For this purpose, both operating elements operable with the hand or also foot switches or input devices such as keyboards or the like, which are operated, for example, by auxiliary personnel during the operation, can be used in the image recording system. Using such input units/input devices, the video can also be provided with additional information by the user, for example, to highlight specific image areas in the video.

It can thus be provided in particular that the image recording system is caused to record the video by means of a recording start user input. In this case, the video can in particular be recorded in a preset advanced imaging mode, thus in particular in a fluorescence imaging mode. Preferably, in this case all parameters are preset during the recording of the video in an image recording mode and therefore defined, so that a user does not have to perform complex manual changes of recording parameters. By means of the recording start user input, the user can therefore start the recording of the video in a preset image recording mode, which deviates from a white light imaging mode used in a normal recording mode. The latter white light imaging mode can in this case be the imaging mode in which the live video is recorded by the system.

It can therefore also be provided that the advanced imaging mode, thus in particular the mentioned fluorescence imaging mode, defines at least two, but preferably all of the following parameters: spatial image resolution; chronological refresh rate during the recording of the video; compression of the video file associated with the video; with respect to the compression of the video, selected parameters of an algorithm used during the recording of the video can also be relevant, since these can influence the later navigation within the video. In addition, however, further parameters can also be defined by the advanced imaging mode, such as: coloration of a fluorescence image display (for example, monochromatic or colored in a false color) and/or extent of the highlighting of edges in the image by means of signal processing and/or application of noise suppression.

The advanced imaging mode, thus in particular the mentioned fluorescence imaging mode, in which the video is recorded, can additionally define at least one, but preferably all of the following parameters: color distribution; image brightness.

In contrast, the replay mode can define the following parameters during the playback of the video retrieved from the memory: refresh rate during the playback and/or speed of the video playback and/or display mode in which the video is displayed.

To make the operation even simpler, it can be provided that the replay user input or the recording user input can be input together with the view mode user input and/or together with the navigation user input via the same manual operating element. Furthermore, it is preferred if such a manual operating element is arranged in a handle area of the endoscope of the image recording system.

Alternatively to one or more manual operating elements on the endoscope, the navigation within the playback of the video in the replay mode, but also the other user inputs, can be carried out, for example, by means of a touch display; this could be arranged, for example, at a central operating unit, in particular at a camera control unit (CCU) of the image recording system. The use of manual operating elements at the endoscope to input said user inputs has the decisive advantage for the surgeon, however, that the hand-eye coordination is improved, the surgeon is distracted less, and the surgeon also does not have to orally instruct a further person to navigate chronologically within the video. Therefore, the usage properties are significantly improved and the rapid inspection and checking of the previously recorded video is greatly facilitated for the surgeon.

It can be provided according to the invention that in the replay mode, the video recorded last chronologically and stored in the memory is always played back. This operating philosophy according to the invention can relieve the user, since this user no longer has to complexly search for the video image file in the internal memory or even on an external server and has to retrieve the video image file, as was the case in previously known approaches. Rather, the last recorded and last stored (for example, in a buffer memory) video is always (automatically) played back in the replay mode. Moreover, preferably both the recording parameters during the recording of the video and also the playback parameters during the playback of this video in the replay mode can be specified beforehand (thus, for example, even before beginning a surgical intervention), so that these parameters no longer have to be manually set by the surgeon during operation.

Upon the storage in the memory, the video can preferably be compressed so that in consideration of the computing power of the image recording system, a delay-free playback of the video without jerky images is possible in the replay mode and at the same time different still images can be displayed at a time interval of less than 1 second (preferably of even less than 500 ms interval). A sufficiently fine chronological resolution during the replay of the video is thus enabled by such a compression, and without jerky images during the playback.

In contrast to previously known concepts, the invention moreover provides that a user can start the playback of the video stored in the memory by changing into the replay mode chronologically independently of the live video image currently recorded by the image recording system and currently displayed. The operation thus becomes more flexible.

The replay mode and also the playback of the stored video can thus be started by a user at arbitrary times. This can take place in particular by ending the recording of the video and/or by means of a/the replay user input and/or, particularly preferably, by changing a/the (current) view mode.

The change into the replay mode will preferably also result here in a change of a display mode and/or an imaging mode: On the one hand, with respect to the display, two video streams now simultaneously have to be displayed (the video loaded from the memory and the live video image). And, on the other hand, the live imaging is no longer to take place in the fluorescent light imaging mode or in an advanced imaging mode, but rather in a white light imaging/WLI mode.

During the recording of videos using the image recording system, in particular in a fluorescence imaging mode or an “advanced imaging mode”, different scenarios are conceivable in principle: (a) For example, it can be reasonable for documentation purposes to record videos at comparatively low resolution in order to limit the memory demand for storing these videos. (b) In contrast, if specific operating situations, for example, for scientific publications, are to be recorded using the system, a particularly high resolution is to be achieved in particular, while the memory demand is then of lower priority. (c) As a third scenario, there is the requirement of recording a flooding procedure with the boundary condition that this video is to be played back reasonably on the image recording system live together with a further live image of the operation scene, which in particular has effects on the required compression and chronological and spatial resolution of the video image data stream to be recorded.

In other words, the recording settings during the recording of the video in a first recording mode can thus be optimized for the replay mode. In order to be able to serve the further scenarios, it can be provided that videos can also be recorded in at least one second mode deviating from the first recording mode. This at least one second recording mode can in particular be designed as part of one specific view mode of a series of different view modes. By selecting the associated view mode, the user can thus put the image recording system into a system state in which the (under certain circumstances respective) second recording mode can be started in order to be able to record a video in the desired settings. The respective view mode can therefore also comprise preset recording parameters which are applied during the recording of videos in this view mode by the system. Both the first recording mode and the at least one second recording mode can be based here on advanced imaging (so that the respective videos are thus not recorded in a white light imaging mode).

Furthermore, as mentioned at the outset, an endoscopic image recording system is provided to achieve the object. This is distinguished in particular in that the endoscope of the image recording system comprises an operating element, using which it is possible to change to a replay mode. If the image recording system is in the replay mode, the system thus displays a video previously recorded using the image sensor and stored in said memory together with a live video image, specifically by means of a method as described above and/or one of the claims directed to a method.

The image recording system according to the invention can additionally comprise at least one display device, on which the video can be displayed simultaneously with the live video image just recorded (either on two separate display devices or on one common display device).

Furthermore, the image recording system according to the invention can be configured to record white light images and at least one type of image deviating therefrom, thus, for example, fluorescence images or images which were recorded in an “advanced imaging mode” (such images typically supply additional image information in comparison to white light images, for example, a local distribution of an oxygen saturation or other parameters which can be optically captured).

The mentioned memory can in particular be designed as an (internal) buffer memory. Live video data which were just recorded using the image recording system, in particular even before a continuous transmission to a display unit, can be buffered in such a buffer memory.

An image recording system according to the invention can have, for example, only a single image sensor. In this case, using this single image sensor, both the video is recorded in an advanced imaging mode (this can in particular be a fluorescence imaging mode using excitation light) and also the live video image, which is then recorded as a white light image in a white light imaging mode using the same image sensor.

In contrast, an alternative embodiment can provide that the image recording system has a first image sensor, which can record said video in an advanced imaging mode, thus, for example, a fluorescence imaging mode. In this case, the image information reaches the first image sensor via a first imaging path. The system can additionally have a second image sensor, which is provided and configured to record white light images via a second imaging path, which deviates spatially from the first imaging path, in particular in the form of live video images or a live video image data stream (live video stream).

### BRIEF DESCRIPTION OF THE DRAWINGS

The invention will now be described in more detail on the basis of exemplary embodiments, but is not restricted to these exemplary embodiments. Further designs of the invention can be obtained from the following description of a preferred exemplary embodiment in conjunction with the general description, the claims, and the drawings.

In the following description of various preferred embodiments of the invention, elements corresponding in their function receive corresponding reference numerals even in the case of differing design or formation.

In the figures:

FIG. 1 shows a first example of an endoscopic image recording system according to the invention, which is currently being used to record white light images in a white light imaging mode,

FIG. 2 shows the image recording system of FIG. 1, wherein now fluorescent light images are recorded using the system in an advanced imaging mode using excitation light,

FIG. 3 shows a schematic view of the display device 8 of the image recording system according to FIGS. 1 and 2, wherein a change of the view mode is illustrated,

FIG. 4 shows a further endoscopic image recording system according to the invention, which, in contrast to that of FIG. 1, only has a single image sensor 3, however, and finally

FIG. 5 shows a further possible embodiment of an endoscopic image recording system according to the invention, which comprises two display devices 8a and 8b and a chip-in-tip (CIT) endoscope.

### DETAILED DESCRIPTION

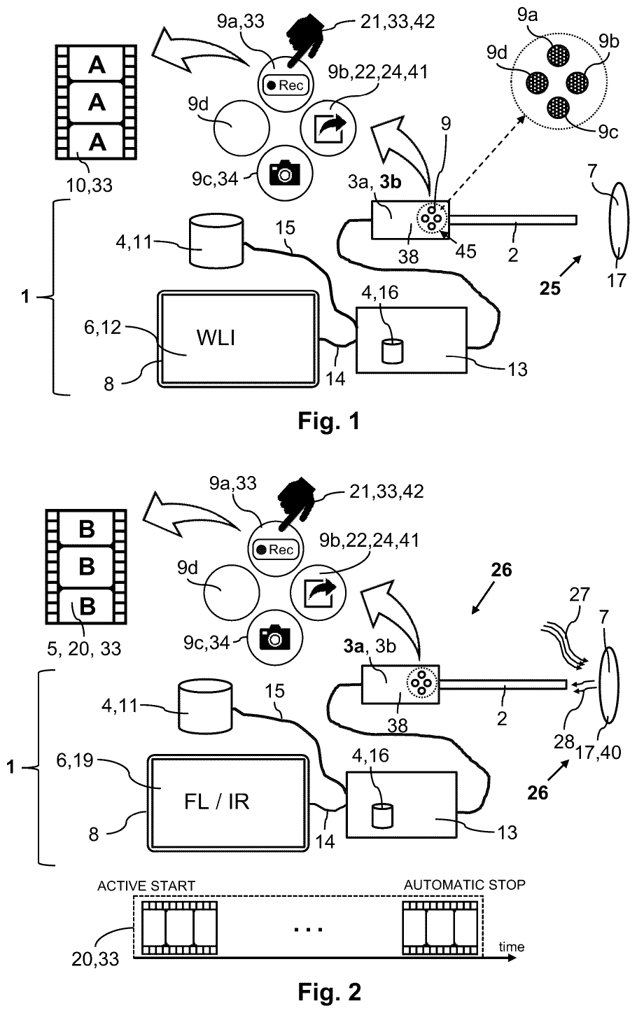

FIG. 1 shows an endoscopic image recording system designated as a whole by 1, which comprises an endoscope 2 having attached camera head 38 having two image sensors 3a and 3b, a camera control unit 13 having an internal buffer memory 16, a display device 8 in the form of a single monitor, and an external memory 11, where the latter is primarily provided to store larger video image files. The schematic data connections 15 shown in FIG. 1 and also the video cable 14 shown could, of course, also be implemented via wireless communication connections. The endoscope 2 shown could also be designed as a video endoscope without influence on the invention, in which at least one image sensor 3 is arranged either in a handle of the endoscope or in a distal end area of the endoscope shaft.

In the situation of FIG. 1, an object 17 is observed using the endoscope 2 and white light images 12 are continuously recorded in the form of a live video image data stream 6 with the aid of the second image sensor 3b using white light illumination. More precisely, these video image data first pass from the endoscope 2 via a data connection to the camera control unit 13, are prepared therein, and before transmission to the display device 8, are buffered in the internal memory 16. In addition, video image files captured in this way can also be stored in the external memory 11 via the data connection 15, which could be implemented, for example, with the aid of a server.

It can furthermore be seen well in FIG. 1 that four manual operating elements 9a to 9d are arranged in the handle area 45 of the endoscope 2 on the camera head 38, using which operating elements the image playback and also different imaging modes can be set.

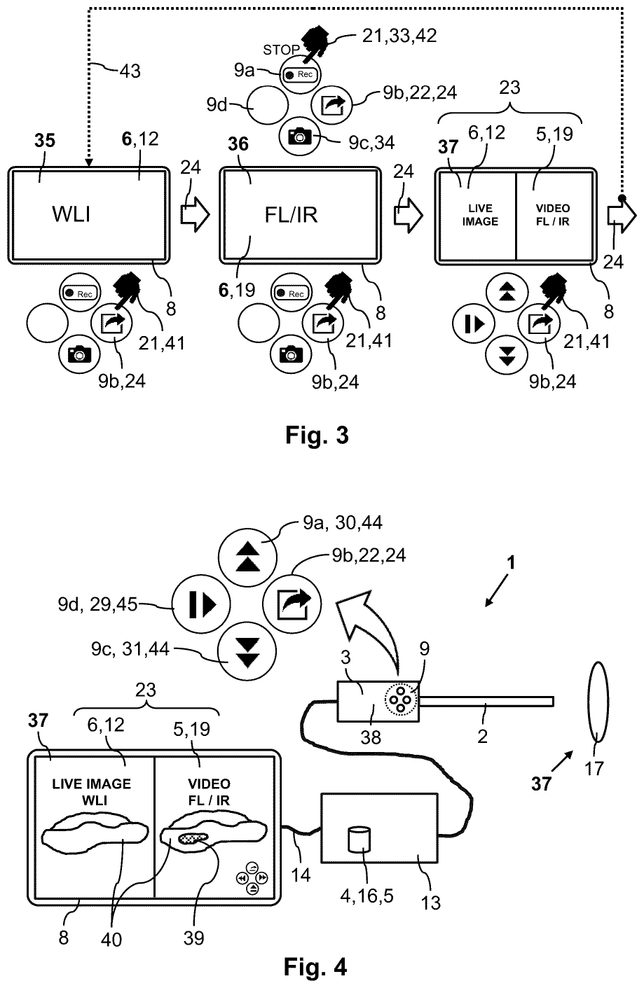

If a user actuates the second operating element 9b, for example, as will be explained in more detail on the basis of FIG. 3, a change 24 of the presently set view mode 35 is performed. By means of such a view mode user input 41, the image recording system 1 can therefore be put into the advanced imaging mode 26 illustrated in FIG. 2, in which an FL (fluorescence image) video stream 20 is now continuously recorded, now with the aid of the first image sensor 3a, which is configured for fluorescent light imaging. For this purpose, a fluorophore 7 is introduced into the tissue 40 observed using the endoscope 2 and the tissue 40 is irradiated using excitation light 27 in the UV wavelength range. In reaction to this optical excitation, the fluorophore 7 emits a fluorescent light 28, which can be sensorially captured with the aid of the first image sensor 3a. Accordingly, a live video image 6 is then likewise displayed on the display device 8 in this situation, but now fluorescent light images 19 are visualized on the monitor.

By manually actuating the first operating element 9a, a user can actuate a recording user input 42 here, which triggers the recording of a video 5 in the fluorescent light imaging mode 26. The video 5 recorded in this manner, which visualizes a flooding procedure performed using the fluorophore 7 in the tissue 40, is stored here in the external memory 11, mediated via the buffer memory 16.

After the operator has successfully recorded the video 5 in this manner and stored it in the memory 4, for example, by pressing the second operating element 9b again, they can cause a renewed change of the view mode and therefore also of the imaging mode and thus change back into the white light imaging mode 25 again, as illustrated in FIG. 1.

However, the user can furthermore also trigger a replay user input 22 by actuating the second operating element 9b, which puts the image recording system 1 into a replay mode 37, as illustrated on the very right in FIG. 3. If the image recording system 1 is in the replay mode 37, on the one hand, a live video image 6 in a white light imaging mode 25, as illustrated in FIG. 1, is continuously recorded using the image recording system 1 and this live video image 6 is displayed on the display device 8. In addition, however, the previously recorded video 5 is loaded from the memory 4 and displayed simultaneously with the live video image 6 in the replay mode 37, wherein this takes place in the example of FIG. 3 by means of a split screen view 23 on the same monitor.

As illustrated in FIG. 2, the video 5 recorded using the image recording system 1 is therefore based on a sensorial capture of fluorescent light 28, which is emitted by the fluorophore 7 in reaction to the optical excitation using the excitation light 27 during the flooding procedure.

In the exemplary embodiment according to FIG. 4, in contrast to those of FIGS. 1 and 2, the single image sensor 3 therein is used to record the WLI live video image 6, but also the video 5 stored in the memory 4. Furthermore, it can be seen that this image recording system 1 according to the invention now has an internal memory 4, 16, from which the video 5 is loaded. The type of display shown in FIG. 4 corresponds to a side-by-side display mode, in which the live video image 6 (which is currently being recorded live in a white light mode) and the video 5 previously recorded in a fluorescence imaging mode are displayed next to one another on the monitor 8.

Furthermore, the situation is illustrated in FIG. 4, in which the image recording system 1 therein is currently in the replay mode 37. It can be seen that the manual operating elements 9a, 9c, and 9d are now assigned deviating functions in comparison to the situation when the image recording system 1 is in a WLI view mode 35 or in an advanced view mode 36 (cf. FIG. 3 left and middle illustration). As soon as the user changes to the replay mode 37, the functional assignment of the manual operating elements 9 therefore changes automatically. In this way, the functional scope reasonable in the respective view mode is always available. This also facilitates the operation during the observation of previously recorded videos 5 in the replay mode 37.

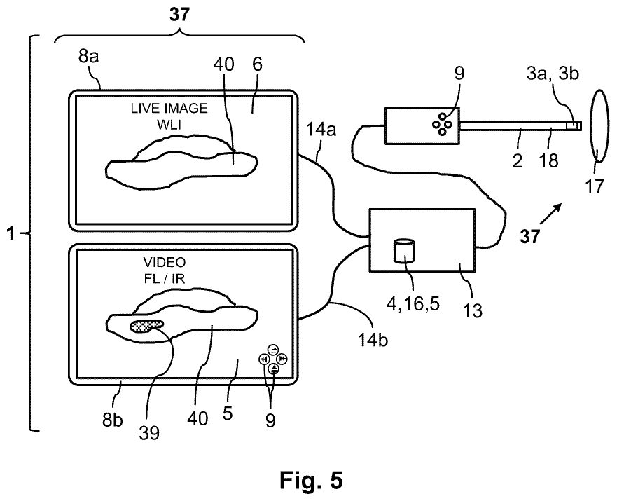

In the exemplary according to FIG. 5, the endoscope 2 therein again has two image sensors 3a and 3b, which are now arranged in a distal tip area of the endoscope 2, however. In other words, this endoscope 2 is therefore designed as a chip-in-tip (CIT) endoscope. In addition, this image recording system 1 according to the invention also has two separate display devices 8a and 8b. Therefore, the previously recorded video 5 and the live video image 6 are played back in parallel but on separate display devices 8a, 8b therein in the replay mode 37. The live video image 6 is thus displayed here on a first monitor 8a and the previously recorded fluorescence video 5 is displayed simultaneously on a second monitor 8b. As illustrated, the endoscopic image recording system 1 is also implemented here with the aid of a chip-in-tip endoscope 2.

The different options for displaying the live video image 6 and the previously recorded video 5 on the respective display device 8, as are shown in the figures, are fundamentally exchangeable here and can be adapted to the desires of the end user according to the requirements of the respective medical intervention.

It can be seen well on the basis of the illustration of FIG. 3 that the replay mode 37 is one view mode of a series made up of three different view modes 35, 36, 37. A user can set the desired view mode via the second operating element 9b by means of a respective view mode user input 21, 41. By repeatedly executing this user input 41, the different view modes are changed in the sequence shown here, specifically in the endless loop 43 illustrated with the aid of the dotted arrow.

The white light view mode 35 illustrated on the very left in FIG. 3 defines, on the one hand, parameters of a white light imaging mode here, which is presently used by the system 1 to generate the live video image 6 and simultaneously an associated display mode in which this live video image 6 is displayed on the monitor. If the user changes to the following advanced view mode 36, the image recording system 1 thus continuously records fluorescent light images 19 in the form of a live video image data stream 6, which is now visualized on the (same) monitor 8 in a second display mode, in a fluorescence imaging mode (which was illustrated in FIG. 2). A renewed actuation of the operating element 9b then leads the user into the replay mode 37, in which the video 5 stored last in the memory 4 is displayed simultaneously with a currently recorded live video image stream 6 on the same monitor 8. The live video image stream 6 is generated by means of white light imaging here in the replay mode.

The great advantage of the simultaneous visualization of the previously recorded video 5 and of the WLI live video image 6 is clear, for example, from FIGS. 4 and 5, in each of which it can be seen that the operator, on the one hand, can continue to observe the scene observed using the endoscope 2 with the aid of the live video image 6 and at the same time at rest and with high chronological resolution can analyze the flooding procedure on the basis of the previously recorded video 5, which is now played back, on the respective display device 8. In the example of FIG. 5, multiple additional operating elements 9 can be seen here on the second display device 8b designed in the form of a touch display. Using these operating elements 9, the surgeon can navigate within the video 5 through respective navigation user inputs 44, for example, fast forward by means of the FF function 30, or fast backward by means of the FB function 31. In this case, the system 1 can display/visualize in images the current functional assignment of the operating elements 9 (designed as graphic elements) to the user likewise on the respective display device 8, in order to thus simplify their use.

During the recording of the video 5, numerous parameters of the advanced imaging mode 26, in which the video 5 is recorded, are previously defined here, in particular the spatial image resolution, the chronological refresh rate, but also the compression of the video file associated with the video 5. At the same time, the user, as soon as they change into the replay mode 37, does not first have to complexly set individual parameters for the playback of the video 5 retrieved from the memory, since these are likewise previously defined, in particular the refresh rate, the speed of the playback, the display mode in which the video 5 is displayed, but also the image brightness.

In the system 1, the user can in this case start a recording of a renewed video 5 by means of a recording user input 42 at any time, wherein the video 5 stored last in the memory 4 is then automatically overwritten. In the replay mode 37, the video 5 stored last in the memory 4 is therefore always played back. Complex searching for and finding the video file is thus avoided, which further improves the usage properties of the system 1.

The change to the replay mode 37 can also be performed at any time by the user, thus in particular independently of the currently used image recording mode or imaging mode.

In the system 1 according to FIG. 5, it is provided that the system 1 changes automatically to the replay mode 37 as soon as the second monitor 8b is connected or as soon as the camera control unit (CCU) 13 recognizes that two monitors 8a, 8b are connected. In contrast, if only one monitor is connected to the CCU 13, the user themselves can change to the replay mode 37, in which then the video 5 and the live video image 6 are played back simultaneously on the then single monitor in a side-by-side view.

As indicated in FIG. 3, a change to the replay mode 37 can take place either as soon as the user themselves actuates a view mode user input 41 via the operating element 9b. Additionally or alternatively, it can also be provided that a change to the replay mode 37 is executed automatically by the system 1 as soon as the recording mode (thus the recording of the video 5) is ended, specifically by the user (in the example by pressing the operating element 9a) or automatically by the system 1 itself (after a defined time span). Such an automatic stop of the recording of the video 5 is illustrated at the lower edge of FIG. 2: the user only has to start the recording here with the aid of the operating element 5a; the system 1 subsequently automatically records a video 5 of preset length (for example, 60 seconds). It can be provided in this case that the user can initially define or change the length of the video to be recorded.

In summary, a simplified approach for a user-friendly playback of a video 5 is provided, which was previously recorded using an endoscopic image recording system 1. To offer the user a high level of security in the use of the endoscopic image recording system 1 and good usage properties at the same time, it is provided that in a replay mode 37, to which the user can change by means of a replay user input 22, for example, on the one hand, a currently recorded live video image 6 is visualized using the image recording system 1 and at the same time the user can observe a playback of the previously recorded video 5 on a display device 8 of the system 1 (cf. FIG. 3).

### LIST OF REFERENCE SIGNS

1 endoscopic image recording system
2 endoscope
3 image sensor
4 memory (internal or external memory)
5 (previously recorded) video
6 live video image
7 fluorophore
8 display device (for example, display or monitor)
9 operating element (preferably manual operating element)
10 WLI (white light imaging) video stream
11 external memory (for WLI image data)
12 white light image (WLI)
13 camera control unit
14 video cable
15 data connection
16 buffer memory
17 object
18 chip-in-tip (CIT) endoscope
19 fluorescent light image (FLI=fluorescence image)
20 FL (fluorescence image) video stream
21 user input
22 replay user input
23 split-screen view
24 change of the view mode
25 white light imaging mode
26 advanced imaging mode (e.g.: FL/IR imaging mode)
27 excitation light
28 fluorescent light
29 start/stop
30 fast forward (FF)
31 fast backward (FB)
32 replay mode (RM)
33 video recording
34 screenshot
35 WLI view mode
36 advanced view mode (in particular FL/IR view mode)
37 replay mode
38 camera head
39 advanced image information
40 tissue/organ
41 view mode user input
42 recording start user input
43 endless loop
44 navigation user input
45 handle area (of 1)

1. A method for user-friendly playback of a video (5), which was previously recorded using an endoscopic image recording system (1), the method comprising:
after recording of the video (5) and storing the video (5) in a memory (4) of the image recording system (1), displaying the previously recorded video (5) in a replay mode (37), including,
loading the previously recorded video (5) from the memory (4) for playback in the replay mode (37); and
in the replay mode (37), simultaneously with the playback of the previously recorded video (5), displaying a live video image (6) currently recorded using the image recording system (1) by the image recording system (1),
wherein the live video image (6) is a white light image (12) and/or is presently being captured in a white light imaging mode (25) using the image recording system (1).

2. The method as claimed in claim 1, wherein the video (5) visualizes a flooding procedure performed using a fluorophore (7) in tissue (40) and/or is recorded in an advanced imaging mode (26) deviating from a white light imaging mode (25), and the loading of the recorded for playback in the replay mode is automatic or in reaction to a replay user input (21, 22).

3. The method as claimed in claim 1, further comprising
capturing the previously recorded video (5) using a first image sensor (3a) of the image recording system (1), and
capturing the live video image (6) using a second image sensor (3a) of the image recording system (1), which is configured for a different type of imaging than the first image sensor imaging,
or
capturing the previously recorded video (5) and the live video image (6) using a same image sensor (3) of the image recording system (1).

4. The method as claimed in claim 3, wherein the capturing step uses the first image sensor (3a) which is configured for advanced imaging, and the second image sensor (3a) which is configured for white light imaging.

5. The method as claimed in claim 1, wherein the previously recorded video (5) and the live video image (6) are displayed to a user in parallel playback on separate display devices (8a, 8b) or on a common display device (8).

6. The method as claimed in claim 1, wherein the previously recorded video (5) is based on a sensorial capture of fluorescent light (28), which is emitted by a fluorophore (7) in reaction to an optical excitation using excitation light (27), or
wherein the recorded video (5) visualizes at least one item of advanced image information (39), which was calculated from sensorially captured image data.

7. The method as claimed in claim 1, wherein
the replay mode (37) is one view mode of a series of at least three different view modes (35, 36, 37), each of which a user can set on the image recording system (1) via a view mode user input (21, 41), and the method further comprising
changing between the different view modes (35, 36, 37) by repeated execution of the view mode user input (21, 41), and
wherein by using a manual operating element (9), it is possible to change between the at least three different view modes (35, 36, 37) of the series, which comprise a white light view mode (35), an advanced view mode (36), and the replay mode (37).

8. The method as claimed in claim 7, wherein each of the view modes (35, 36, 37) of the series comprises and defines
an imaging mode, which is currently used to generate the live video image (6), and
a display mode, in which the live video image (6) is visualized on a display device (8),
and/or
wherein
the white light view mode (35) defines parameters of the white light imaging mode (25),
the advanced view mode (36) defines parameters of an advanced imaging mode (26), comprising a fluorescence imaging mode or an imaging mode which enables a visualization of an item of advanced image information (39), and
the replay mode (37) defines parameters of the white light imaging mode (25) for the live video image (6) and a display mode for parallel playback of the video (5) and the live video image (6).

9. The method as claimed in claim 1, further comprising a user using a navigation user input (44) to navigate in the replay mode (37), in order to have different time segments of the video (5) displayed,
wherein the navigation user input (44) allows jumping forward or back by a defined time span in the video (5) while the previously recorded video (5) is played back in the replay mode (37) in a video endless loop.

10. The method as claimed in claim 7, further comprising the image recording system (1) including a recording start user input (42), and starting the recording of the video (5) by activating the recording start user input (42) in a preset advanced imaging mode (26), and the advanced imaging mode (26) defines at least two of the following parameters:
a spatial image resolution;
a chronological refresh rate during the recording;
a compression of the video file associated with the video (5);
a color distribution; or
an image brightness.

11. The method as claimed in claim 1, wherein the replay mode (37) defines at least two of the following parameters during the playback of the previously recorded video (5) retrieved from the memory (4):
a refresh rate during the playback;
a speed of the video playback; or
a display mode in which the previously recorded video (5) is displayed.

12. The method as claimed in claim 10, further comprising
inputting a replay user input (22) or the recording start user input (42)
together with the view mode user input (41) and/or with a navigation user input (44) that is configured to navigate in the replay mode (37), via a same manual operating element (9b), which is arranged in a handle area (45) of an endoscope (2) of the image recording system (1).

13. The method as claimed in any claim 1, wherein in the replay mode (37), the chronologically last recorded video (5) stored in the memory (4) is always played back.

14. The method as claimed in claim 1, further comprising compressing the previously recorded video (5) during storage in the memory (4) so that based on a computing power of the image recording system (1), a delay-free playback of the video (5) without jerky images is provided in the replay mode (37) and at a same time different still images are adapted to be displayed with a time interval of less than 1 second.

15. The method as claimed in claim 1, further comprising starting a playback of the previously recorded video (5) stored in the memory (4) by a user by changing to the replay mode (37) independent in time of the live video image (6) currently recorded by the image recording system (1) and displayed.

16. The method as claimed in claim 7, wherein the replay mode (37) and the playback of the video (6) are adapted to be started by a user by at least one of:
ending the recording of the video (5),
a replay user input (22), or
by changing the view mode (35, 36, 37).

17. An endoscopic image recording system (1), comprising an endoscope (2);
at least one image sensor (3) configured for sensorial capture of fluorescent light (28) and/or an item of advanced image information (39); and
a memory (4) for storing videos (5), which are recorded using the at least one image sensor (3); and
an operating element (9b), configured to change to a replay mode (37), in which the image recording system (1) displays one of the videos (5) previously recorded using the image sensor (3) and stored in the memory (4) together with a live video image (6) using the method according to claim 1.

18. The image recording system (1) as claimed in claim 17, wherein the memory (4) comprises a buffer memory (16), in which live video data from the image recording system (1) is adapted to be buffered before a continuous transmission to a display unit (8).

19. The image recording system (1) as claimed in claim 17, wherein the image recording system (1) has only a single said image sensor (3), that is configured to capture the previously recorded video (5) in the advanced imaging mode (26), and to record the live video image (6) as a white light image (12) in a white light imaging mode (25).

20. The image recording system (1) as claimed in claim 17, wherein the at least one image sensor (3) comprises:
a first image sensor (3a), which is adapted record the video (5), in a fluorescence imaging mode or an advanced imaging mode (26), via a first imaging path, and
a second image sensor (3b), which is adapted to record white light images (12) as a live video image (6) via a second imaging path spatially deviating from the first imaging path.
